(19) United States
(12) Patent Application Publication
(10) Pub. No.: US 2020/0352938 A1
(43) Pub. Date: Nov. 12, 2020

(54) PHARMACEUTICAL COMPOSITIONS FOR TREATING PRESBYOPIA AND METHODS FOR FABRICATING THEREOF

(71) Applicant: Harrow IP, LLC, Nashville, TN (US)

(72) Inventor: Dennis Elias Saadeh, Brentwood, TN (US)

(21) Appl. No.: 16/858,377

(22) Filed: Apr. 24, 2020

Related U.S. Application Data
(60) Provisional application No. 62/845,061, filed on May 8, 2019.

(51) Int. Cl. A61K 31/498; A61K 47/10; A61K 47/36; A61K 31/14; A61K 45/06; A61K 31/496; A61K 47/34; A61K 9/00

(52) U.S. Cl. CPC A61K 31/498; A61K 47/10; A61K 47/36; A61K 9/0048; A61K 45/06; A61K 31/496; A61K 47/34; A61K 31/14

(57) ABSTRACT

Pharmaceutical compositions for treating presbyopia are described, the compositions comprising choline esterase inhibitor(s) and several other components such as α-1-adrenergic antagonist(s), non-steroid anti-inflammatory drug(s), and/or adrenergic antagonist(s). Methods for fabricating the compositions and using the compositions are also described.

### CROSS REFERENCE TO RELATED APPLICATIONS

This application claims the benefit of priority under 35 U.S.C. § 119(e) to U.S. Provisional Application No. 62/845,061, filed on May 8, 2019, the entire content of which is hereby incorporated by reference.

### FIELD OF THE INVENTION

The present invention relates generally to the field of pharmaceuticals and more specifically to ophthalmic pharmaceutical compositions for treating presbyopia, and to methods of preparing and using such compositions.

### BACKGROUND

Presbyopia is caused by the process of aging of the eye and commonly results in progressively worsening ability to focus clearly on close objects. More specifically, hardening of the lens of the eye causes the eye to focus light behind, rather than on, the retina when looking at close objects. In other words, it is a condition in which incoming light focuses behind the retina, leading to difficulty focusing on close-up objects.

Typically, presbyopia is treated by eyesight correction, i.e., by using corrective lenses or eyeglasses such as reading glasses. Surgical treatments are also possible in some cases. It has been long felt that there is a need to treat presbyopia without using eyeglasses or surgery, i.e., pharmaceutically. However, no pharmaceutical treatment of presbyopia has been approved thus far, and accordingly, this need remains unfulfilled.

It is, therefore, desirable to have pharmaceutical compositions that may be used for safe and efficient treatment of presbyopia. This patent specification discloses such pharmaceutical compositions that can achieve such positive patient outcomes, and methods of fabricating and administering the same.

### SUMMARY

According to various embodiments of the invention, an ophthalmological pharmaceutical composition is provided for treating, alleviating, and/or mitigating presbyopia, the composition comprising, consisting of, or essentially consisting of, a therapeutically effective quantity of a cholinesterase inhibitor, a therapeutically effective quantity of an α-1-adrenergic antagonist, and/or a therapeutically effective quantity of an α-2-adrenergic agonist, and a quantity of de-ionized water, and may further optionally include a non-steroid anti-inflammatory drug, a penetration enhancer, glycerol, dextran and/or chondroitin sulfate.

According to various embodiments of the invention, the presence of a non-steroid anti-inflammatory drug in the ophthalmological pharmaceutical composition is required in addition to the presence of a cholinesterase inhibitor, but the presence of α-1-adrenergic antagonist is optional.

According to various embodiments of the invention, the presence of an adrenergic agonist is required in addition to the presence of a cholinesterase inhibitor, but the presence of an α-1-adrenergic antagonist and/or a non-steroid anti-inflammatory drug in the ophthalmological pharmaceutical composition is optional.

According to various embodiments of the invention, methods for treating, alleviating, and/or mitigating presbyopia in a human subject are provided, where the methods comprise administering to the subject the above-described ophthalmological pharmaceutical composition via eye drops into one eye or both eyes.

### DETAILED DESCRIPTION

### A. Terms and Definitions

Unless specific definitions are provided, the nomenclatures utilized in connection with, and the laboratory procedures and techniques of analytical chemistry, synthetic organic and inorganic chemistry described herein, are those known in the art. Standard chemical symbols are used interchangeably with the full names represented by such symbols. Thus, for example, the terms “hydrogen” and “H” are understood to have identical meaning. Standard techniques may be used for chemical syntheses, chemical analyses, formulating compositions and testing them. The foregoing techniques and procedures can be generally performed according to conventional methods well known in the art.

It is to be understood that both the foregoing general description and the following detailed description are exemplary and explanatory only and are not restrictive of the invention claimed. As used herein, the use of the singular includes the plural unless specifically stated otherwise. The section headings used herein are for organizational purposes only and are not to be construed as limiting the subject matter described.

As used herein, “or” means “and/or” unless stated otherwise. Furthermore, use of the term “including” as well as other forms, such as “includes,” and “included,” is not limiting.

“About” as used herein means that a number referred to as “about” comprises the recited number plus or minus 1-10% of that recited number. For example, “about” 100 degrees can mean 95-105 degrees or as few as 99-101 degrees depending on the context. Whenever it appears herein, a numerical range such as “1 to 20” refers to each integer in the given range; i.e., meaning only 1, only 2, only 3, etc., up to and including only 20, as well as to the numbers in between integers, e.g., 1.5 or 2.5, and the like.

The term “pharmaceutical composition” is defined as a chemical or a biological compound or substance, or a mixture or combination of two or more such compounds or substances, intended for use in the medical diagnosis, cure, treatment, or prevention of disease or pathology.

The term “presbyopia” is defined for the purposes of the present application as a visual condition which typically first becomes apparent in a person's middle age in which loss of elasticity of the lens of the eye causes defective accommodation and an inability or a reduction in the ability to focus sharply for near vision.

The term “cholinesterase inhibitor” is defined for the purposes of the present application as a chemical compound that inhibits the acetylcholinesterase enzyme from cleaving acetylcholine. Reversible, irreversible, and quasi-irreversible inhibitors are within the scope of the present invention.

The term “α-1-adrenergic antagonist” (inclusive of both selective and non-selective α-1-adrenergic antagonists) is defined for the purposes of the present application as a compound belonging to the group of chemical substances that reduce the effect of α-1-adrenergic receptors. It is specifically provided that α-1-adrenergic antagonists are to be distinguished from, and not to be confused with, all other kinds of adrenergic antagonists, such as α-2-adrenergic antagonists and all kinds of β-adrenergic antagonists. Only α-1-adrenergic antagonists are intended to be within the scope of the present invention.

The term “adrenergic agonist” is defined for the purposes of the present application as a compound belonging to the group of chemical substances that stimulate a response from the adrenergic receptors, i.e., G-protein coupled receptors activating signal transduction pathways. All adrenergic agonists, including their sub-classes and sub-types, are intended to be within the scope of the present invention.

The term “non-steroid anti-inflammatory drug” refers to substances or compounds that are free of steroid moieties and provide analgesic, antipyretic and/or anti-inflammatory effects.

The term “excipient” is defined for the purposes of the present application as a pharmacologically inactive substance that is formulated in combination with a pharmacologically active ingredient of the pharmaceutical composition, and is inclusive of bulking agents, fillers, diluents and products used for facilitating drug absorption or solubility, or for other pharmacokinetic considerations.

The term “carrier” refers to a substance that serves as a vehicle for improving the efficiency of delivery and the effectiveness of a pharmaceutical composition.

The term “therapeutically effective amount” is defined as the amount of a compound or pharmaceutical composition that will elicit the biological or medical response of a tissue, system, animal or human that is being sought by the researcher, medical doctor or other clinician.

The term “pharmaceutically acceptable” when used to define a carrier, whether diluent or excipient, refers to a substance that is compatible with the other ingredients of the formulation and not deleterious to the recipient thereof.

The terms “administration of a composition” or “administering a composition” are defined to include an act of providing a compound of the invention or pharmaceutical composition to the subject in need of treatment.

### B. Embodiments of the Invention

The present specification discloses ophthalmic pharmaceutical compositions for treating, alleviating, and/or mitigating presbyopia as well as methods that can be used to effectuate such treatments. According to certain embodiments of the present invention, pharmaceutical compositions intended for such treatments comprise, consist of, or consist essentially of:
(1) a therapeutically effective quantity of one or a combination of several cholinesterase inhibitor(s);
(2) a therapeutically effective quantity of one or a combination of several α-1-adrenergic antagonist(s) or a therapeutically effective quantity of one or a combination of several non-steroid anti-inflammatory drug(s), wherein:

if an α-1-adrenergic antagonist is used in the composition, the use of a non-steroid anti-inflammatory drug is optional, and vice versa, i.e., if a non-steroid anti-inflammatory drug is used in the composition, then the use of an α-1-adrenergic antagonist is optional. Expressed differently, the composition may include either an α-1-adrenergic antagonist or a non-steroid anti-inflammatory drug, or both may be used if desired;
(3) optionally, an excipient comprising, consisting of, or consisting essentially of, one or several penetration enhancer(s); a quantity of glycerol, a quantity of dextran, a quantity of chondroitin sulfate, or combinations thereof; and
(4) a quantity of de-ionized water.

The concentration of a cholinesterase inhibitor(s) in the pharmaceutical compositions described herein may be between about 0.0001% (w/v) and about 0.25% (w/v), such as between about 0.001% (w/v) and about 0.1% (w/v), for example, about 0.005% (w/v), 0.01% (w/v), 0.015% (w/v), 0.02% (w/v), 0.025% (w/v), or 0.03% (w/v), preferably about 0.03% (w/v).

Non-limiting examples of cholinesterase inhibitor(s) that may be used in the compositions of the present inventions include echothiophate iodide (also known as phospholine iodide), physostigmine, pyridostigmine, neostigmine, aceclidine, ambenonium, demecarium, rivastigmine, galantamine, caffeine, rosmarinic acid, α-pinene, donepezil, tacrine, edrophonium, huperzine A, ladostigil, ungeremine, lactucopicrin, acotiamide, diisopropyl fluorophosphate, cadusafos, chlorpyrifos, cyclosarin, dichlorvos, dimethoate, metrifonate, and any combination thereof.

Those having ordinary skill in the art may use other cholinesterase inhibitor(s) in addition to, or instead of, those enumerated above but may find that, out of the inhibitors listed above, use of the five inhibitors shown in Table 1, below, is particularly beneficial.

TABLE 1
Cholinesterase Inhibitor(s)

| Compound | IUPAC Name | Chemical Structure |
|---|---|---|
| Echothiophate iodide | 2- (diethoxyphosphorylsulfanyl)ethyl- N,N,N-trimethylazanium iodide | 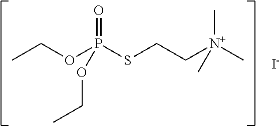 |
| Aceclidine | 1-azabicyclo[2.2.2]octan-3-yl acetate | 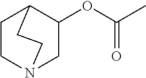 |
| Physostigmine | [(3aR,8bS)-3,4,8b-trimethyl-2,3a- dihydro-1H-pyrrolo[2,3-b]indol-7- yl] N-methylcarbamate | 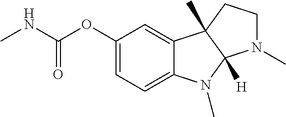 |
| Pyridostigmine | (1-methylpyridin-1-ium-3-yl) N,N- dimethylcarbamate | 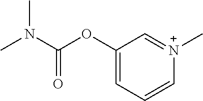 |
| Neostigmine | [3- (dimethylcarbamoyloxy)phenyl]- trimethylazanium | 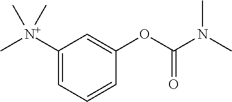 |

The concentration of an α-1-adrenergic antagonist, if used in the pharmaceutical compositions described herein, may be between about 0.01% (w/v) and about 0.5% (w/v), such as between about 0.2% (w/v) and about 0.4% (w/v), for example, about 0.25% (w/v), about 0.3% (w/v), or about 0.35% (w/v), preferably about 0.25% (w/v).

If an α-1-adrenergic antagonist is used in the composition, specific examples of such acceptable antagonists include, without limitation, dapiprazole, phentolamine, phentolamine mesylate, tamsulosin, alfuzosin, doxazosin, prazosin, terazosin, and any combination thereof. Those having ordinary skill in the art may find use of dapiprazole particularly beneficial. The chemical structure of dapiparazol (IUPAC, 3-[2-[4-(2-methylphenyl)piperazin-1-yl]ethyl]-5,6,7,8-tetrahydro-[1,2,4]triazolo[4,3-a]pyridine) is shown below:

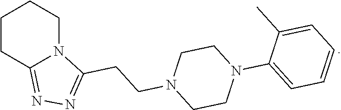 [SMILES: Cc1ccccc1N1CCN(CCc2nnc3n2CCCC3)CC1]

If desired, one or several additional α-1-adrenergic antagonist(s) may be used in combination with, or instead of, any of dapiprazole, tamsulosin, alfuzosin, doxazosin, prazosin, or terazosin. Illustrative, but non-limiting, examples of such additional antagonist(s) that may be used include non-selective α-1-adrenergic antagonist(s), e.g., phentolamine, phentolamine mesylate, phenoxybenzamine, tolazoline, and trazodone.

If used, the concentration of the non-steroid anti-inflammatory drug(s) in the ophthalmological compositions of the present application may be between about 0.01% (w/v) and about 0.5% (w/v), for example, about 0.05% (w/v), about 0.1% (w/v), about 0.15% (w/v), about 0.2% (w/v), or about 0.25% (w/v), preferable about 0.25% (w/v). Non-limiting examples of the non-steroid anti-inflammatory drug(s) that may be utilized include bromfenac, ketorolac, diclofenac, nepafenac, and combinations thereof.

In various embodiments, adrenergic agonist(s) may be optionally used in combination with, or instead of, α-1-adrenergic antagonist(s) and/or non-steroid anti-inflammatory drug(s), in concentrations between about 0.01% (w/v) and about 0.5% (w/v), such as between about 0.1% (w/v) and about 0.3% (w/v), for example, about 0.15% (w/v), about 0.2% (w/v), or about 0.25% (w/v), preferably about 0.15% (w/v). Some examples of acceptable adrenergic agonist(s) include, without limitation, apraclonidine and brimonidine.

As stated above, the ophthalmological compositions of the present application may further optionally include an excipient which may comprise one or several penetration enhancer(s). If penetration enhancers are so used, they may be present in the composition in the concentration between about 0.1% (w/v) and about 20.0% (w/v).

One suitable penetration enhancer that may be employed is a non-ionic polyoxyethlene-polyoxypropylene block copolymer which may be present in the composition in the concentration between about 0.1% (w/v) and about 0.5% (w/v). This block copolymer has the following general structure:

HO—(CH₂—CH₂—O)ₓ—(C₃H₆—O)_{y}—(CH₂—CH₂—O)ₓ—H,

wherein in the chemical structure above x is an integer having the value of at least 8 and y is an integer having the value of at least 38. Polyoxyethlene-polyoxypropylene block copolymer(s) that can be used may be those belonging to the PLURONIC® or POLOXAMER® families, chemically, poly(ethylene glycol)-block-poly(propylene glycol)-block-poly(ethylene glycol), both available from BASF Corp. and from several other vendors and having the following general chemical structure:

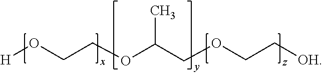

Another specific and non-limiting example of a non-ionic polyoxyethlene-polyoxypropylene block copolymer that can be used is the product known under the trade name PLURONIC® L64, which is described by the chemical structure above, with the molecular weight of the polyoxypropylene portion of about 1,750 Daltons, about a 40% polyoxyethylene content (mass), and the average overall molecular weight of about 2,900 Daltons.

Another specific non-limiting example of a non-ionic polyoxyethlene-polyoxypropylene block copolymer that can be used is the product known under the trade name Poloxamer 407® (also known as PLURONIC® F127), which is also described by the chemical structure above, with the molecular weight of the polyoxypropylene portion of about 4,000 Daltons, about a 70% polyoxyethylene content (mass), the overall molecular weight of between about 9,840 Daltons and about 14,600 Daltons.

One non-limiting example of other penetration enhancer(s) that may be used is polyacrylic acid cross-linked with divinyl glycol, with calcium as a counter-ion, in a concentration of between about 0.5% (w/v) and about 1.0% (w/v). This product which is known under the trade name NOVEON® Polycarbophil (Lubrizol Corp., Wickliffe, Ohio) has the following general chemical structure:

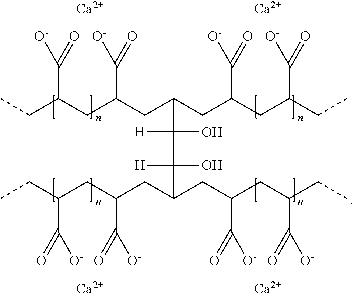

Those having ordinary skill in the art may select (an)other or additional compound(s) as penetration enhancer(s), if desired, as well as the quantity thereof.

As mentioned above, the excipient may also include additional compounds if necessary, e.g., glycerol, dextran, chondroitin sulfate, or any combination thereof.

According to various embodiments, methods for fabricating the above-described pharmaceutical compositions are provided. A one-batch formulation method may be used, where the components of the pharmaceutical formulation can be combined in a single container; the components may be added to the container simultaneously or consecutively. Those having ordinary skill in the art can choose the best method for preparing the compositions.

Pharmaceutical compositions prepared as described above can be used to treat, prevent or alleviate presbyopia and can be normally delivered via eye drops. An ordinarily skilled physician may prescribe a patient-specific dosage. It will be understood by those having ordinary skill in the art that the specific dose level and frequency of dosage for any particular patient may be varied and will depend upon a variety of factors including the activity of the specific compound employed, the metabolic stability and length of action of that compound, the age, body weight, general health, gender, diet, and the severity of the particular disease or condition being treated. In purely exemplary embodiments, the dosage may be 1 to 2 drops that may be administered 1 to 2 times daily, and the drops may be administered either to both eyes of the patient or only to 1 eye, as appropriate.

In additional embodiments, pharmaceutical kits are provided. The kit includes a sealed container approved for the storage of pharmaceutical compositions, the container containing one of the above-described pharmaceutical compositions. An instruction for the use of the composition along with information about the composition are to be included in the kit.

The following examples are provided to further elucidate the advantages and features of the present invention but are not intended to limit the scope of the invention. The examples are for illustrative purposes only. USP pharmaceutical grade products were used in preparing the formulations described below.

### C. Examples

### Example 1. Preparing a Pharmaceutical Composition No. 1

A pharmaceutical composition may be prepared as described below. The following products may be used in the amounts specified:

(1) about 0.03 g of ecothiophate iodide powder;

(2) about 0.5 g of dapiprazole powder;

(3) about 0.4 g of ketorolac tromethamine powder;

(4) about 0.8 g of potassium acetate powder;

(5) about 0.25 g of chondroitin sulfate sodium powder;

(6) about 0.25 g of dextran 40 powder;

(7) about 1.0 mL of POLYSORBATE® 80;

(8) about 0.5 mL of glycerin;

(9) about 0.2 g of POLOXAMER®407;

(10) about 0.003 g of benzalkonium chloride;

(11) glacial acetic acid solution or sodium hydroxide, for adjusting pH; and

(12) about 100 mL of sterile water.

In a sterilized beaker with spin bar that is filled with about 90% of the required sterile water, ecothiophate iodide, dapiprazole, ketorolac tromethamine, potassium acetate, chondroitin sulfate sodium, and dextran 40 powders may be added one by one with continuous spinning at room temperature until all the powders are dissolved. Then, glycerin, POLYSORBATE® 80 and benzalkonium chloride may be added with continued spinning, followed by adjusting pH to about 6.5 using acetic acid or sodium hydroxide, whichever applies, and by adding POLOXAMER®407.

The value of pH can then be again adjusted and maintained at about 6.5±0.1, and the remaining quantity of sterile water may be then added. The final product may then be filtered through a 0.22 μm filter into a sterile dispensing container, packaged at 5 mL fill with 0.2 mL overfill, into pre-sterilized 3-piece dropper bottles and a label may be affixed to the container.

### Example 2. Preparing a Pharmaceutical Composition No. 2

A pharmaceutical composition may be prepared as described below. The following products may be used in the amounts or concentrations specified:

(1) physostigmine, at about 0.002% (w/v);

(2) dapiprazole, at about 0.25% (w/v);

(3) sodium acetate trihydrate, at about 0.2% (w/v);

(4) POLYCARBOPHIL®, at about 0.5% (w/v);

(5) glacial acetic acid solution or sodium hydroxide, for adjusting pH; and

(6) about 100 mL of sterile water.

Phase I aqueous polyacrylic acid suspension is made with Polycarbophil (Noveon A-A-1) and sterile water. Phase I is then autoclaved under sterile conditions at 121° C. and 17-18 psi of pressure for 30 minutes. Phase II is prepared by adding all of the remaining ingredients one by one until dissolved in sterile water. Phases I and II are then combined with mixing under aseptic conditions. The pH of the resulting solution is then adjusted with sterile filtered glacial acetic acid to between 6.2 and 6.4. The mixing will continue for a minimum of 15 minutes and a maximum of 1 hour. The combined solution is then brought to final weight with sterile water for injection. The suspension is then packaged at 5 mL fill with 0.2 mL overfill, into pre-sterilized 3-piece dropper bottles and a label may be affixed to the bottles.

### Example 3. Preparing a Pharmaceutical Composition No. 3

A pharmaceutical composition may be prepared as described below. The following products may be used in the amounts or concentrations specified:

(1) neostigmine, at about 0.02% (w/v);

(2) brimonidine tartarate, at about 0.1% (w/v);

(3) diclofenac sodium, at about 0.1% (w/v);

(4) about 0.25 g of sodium acetate powder;

(5) about 0.03 g of citric acid;

(6) POLOXAMER®407, at about 0.5% (w/v);

(7) glacial acetic acid solution or sodium hydroxide, for adjusting pH; and

(8) about 100 mL of sterile water.

The product can be then made by mixing components (1) through (6), above, and using the procedure outlined in Example 1, including the value of pH to be maintained at about 6.5±0.1, as described in Example 1.

Although the invention has been described with reference to the above examples, it will be understood that modifications and variations are encompassed within the spirit and scope of the invention. Accordingly, the invention is limited only by the following claims.

What is claimed is:

1. An ophthalmological pharmaceutical composition for treating, alleviating, and/or mitigating presbyopia, the composition comprising:
(a) a therapeutically effective quantity of at least one cholinesterase inhibitor;
(b) a therapeutically effective quantity of at least one α-1-adrenergic antagonist;
(c) optionally, a therapeutically effective quantity of at least one non-steroid anti-inflammatory drug;
(d) optionally, at least one penetration enhancer;
(e) optionally, a quantity of glycerol;
optionally, a quantity of dextran;
(g) optionally, a quantity of chondroitin sulfate; and
(h) a quantity of de-ionized water.

2. The ophthalmological pharmaceutical composition of claim 1, wherein the cholinesterase inhibitor is selected from the group consisting of echothiophate iodide, physostigmine, pyridostigmine, neostigmine, aceclidine, ambenonium, demecarium, rivastigmine, galantamine, caffeine, rosmarinic acid, α-pinene, donepezil, tacrine, edrophonium, huperzine A, ladostigil, ungeremine, lactucopicrin, acotiamide, diisopropyl fluorophosphate, cadusafos, chlorpyrifos, cyclosarin, dichlorvos, dimethoate, metrifonate, and any combination thereof.

3. The ophthalmological pharmaceutical composition of claim 2, wherein the cholinesterase inhibitor is selected from the group consisting of echothiophate iodide, physostigmine, pyridostigmine, neostigmine, and any combination thereof.

4. The ophthalmological pharmaceutical composition of claim 2, wherein the cholinesterase inhibitor is neostigmine.

5. The ophthalmological pharmaceutical composition of claim 1, wherein the α-1-adrenergic antagonist is selected from the group consisting of dapiprazole, tamsulosin, alfuzosin, doxazosin, prazosin, terazosin, phentolamine, phentolamine mesylate, phenoxybenzamine, tolazoline, and trazodone, and any combination thereof.

6. The ophthalmological pharmaceutical composition of claim 5, wherein the α-1-adrenergic antagonist is dapiprazole.

7. The ophthalmological pharmaceutical composition of claim 1, wherein the non-steroid anti-inflammatory drug is selected from the group consisting of bromfenac, ketorolac, diclofenac, nepafenac, and combinations thereof.

8. The ophthalmological pharmaceutical composition of claim 1, wherein the penetration enhancer is selected from the group consisting of non-ionic polyoxyethlene-polyoxypropylene block copolymers, cross-linked polyacrylic acid, and combinations thereof.

9. The ophthalmological pharmaceutical composition of claim 8, wherein the penetration enhancer is poly(ethylene glycol)-block-poly(propylene glycol)-block-poly(ethylene glycol).

10. The ophthalmological pharmaceutical composition of claim 1, wherein the concentration of the cholinesterase inhibitor(s) in the composition is between about 0.0001% (w/v) and about 0.25% (w/v), and the concentration of the α-1-adrenergic antagonist(s) is between about 0.1% (w/v) and about 0.5% (w/v).

11. A method for treating, alleviating, and/or mitigating presbyopia in a human subject in need of such treatment, comprising administering to the subject the ophthalmological pharmaceutical composition of claim 1, thereby treating, alleviating, and/or mitigating presbyopia.

12. The method of claim 11, wherein the ophthalmological pharmaceutical composition is administered by eye drops.

13. The method of claim 11, wherein the ophthalmological pharmaceutical composition is administered into one eye of the subject, or to both eyes of the subject.

14. An ophthalmological pharmaceutical composition for treating, alleviating, and/or mitigating presbyopia, the composition comprising:
(a) a therapeutically effective quantity of a cholinesterase inhibitor selected from the group consisting of echothiophate iodide, physostigmine, pyridostigmine, neostigmine, aceclidine, ambenonium, demecarium, rivastigmine, galantamine, caffeine, rosmarinic acid, α-pinene, donepezil, tacrine, edrophonium, huperzine A, ladostigil, ungeremine, lactucopicrin, acotiamide, diisopropyl fluorophosphate, cadusafos, chlorpyrifos, cyclosarin, dichlorvos, dimethoate, metrifonate, and any combination thereof;
(b) a therapeutically effective quantity of a non-steroid anti-inflammatory drug selected from the group consisting of bromfenac, ketorolac, diclofenac, nepafenac, and any combination thereof;
(c) optionally, a therapeutically effective quantity of an α-1-adrenergic antagonist selected from the group consisting of dapiprazole, phentolamine, phentolamine mesylate, tamsulosin, alfuzosin, doxazosin, prazosin, terazosin, and any combination thereof;
(d) optionally, at least one penetration enhancer selected from the group consisting of non-ionic polyoxyethlene-polyoxypropylene block copolymers, cross-linked polyacrylic acid, and any combination thereof;
(e) optionally, a quantity of glycerol;
optionally, a quantity of dextran;
(g) optionally, a quantity of chondroitin sulfate; and
(h) a quantity of de-ionized water.

15. The ophthalmological pharmaceutical composition of claim 14, wherein the cholinesterase inhibitor is selected from the group consisting of echothiophate iodide, physostigmine, pyridostigmine, neostigmine, aceclidine and any combination thereof.

16. The ophthalmological pharmaceutical composition of claim 14, wherein the penetration enhancer is poly(ethylene glycol)-block-poly(propylene glycol)-block-poly(ethylene glycol).

17. An ophthalmological pharmaceutical composition for treating, alleviating, and/or mitigating presbyopia, the composition comprising:
(a) a therapeutically effective quantity of a cholinesterase inhibitor selected from the group consisting of echothiophate iodide, physostigmine, pyridostigmine, neostigmine, ambenonium, demecarium, rivastigmine, galantamine, caffeine, rosmarinic acid, α-pinene, donepezil, tacrine, edrophonium, huperzine A, ladostigil, ungeremine, lactucopicrin, acotiamide, diisopropyl fluorophosphate, cadusafos, chlorpyrifos, cyclosarin, dichlorvos, dimethoate, metrifonate, and any combination thereof;
(b) a therapeutically effective quantity of an adrenergic agonist selected from the group consisting of apraclonidine, brimonidine, and any combination thereof;
(c) optionally, a therapeutically effective quantity of a non-steroid anti-inflammatory drug selected from the group consisting of bromfenac, ketorolac, diclofenac, nepafenac, and any combination thereof;
(d) optionally, a therapeutically effective quantity of an α-1-adrenergic antagonist selected from the group consisting of dapiprazole, tamsulosin, alfuzosin, doxazosin, prazosin, terazosin, phentolamine, phentolamine mesylate, phenoxybenzamine, tolazoline, and trazodone, and any combination thereof;
(e) optionally, at least one penetration enhancer selected from the group consisting of non-ionic polyoxyethlene-polyoxypropylene block copolymers, cross-linked polyacrylic acid, and any combination thereof;
(f) optionally, a quantity of glycerol;
(g) optionally, a quantity of dextran;
(h) optionally, a quantity of chondroitin sulfate; and
(i) a quantity of de-ionized water.

18. The ophthalmological pharmaceutical composition of claim 17, wherein the cholinesterase inhibitor is selected from the group consisting of echothiophate iodide, physostigmine, pyridostigmine, neostigmine, and any combination thereof.

19. The ophthalmological pharmaceutical composition of claim 17, wherein the adrenergic agonist is brimonidine.

20. The ophthalmological pharmaceutical composition of claim 17, wherein the penetration enhancer is poly(ethylene glycol)-block-poly(propylene glycol)-block-poly(ethylene glycol).
